(19) United States
(12) Patent Application Publication
(10) Pub. No.: US 2010/0318088 A1
(43) Pub. Date: Dec. 16, 2010

(54) METHODS AND DEVICES FOR DEPLOYING BIOLOGICAL IMPLANTS

(75) Inventors: James Brian Warne, Los Gatos, CA (US); Kirk Davis, Los Gatos, CA (US); Craig J. Corey, Hillsborough, CA (US); Roderick James Pimlott, Atherton, CA (US); George Yoseung Choi, Menlo Park, CA (US)

Correspondence Address: LEVINE BAGADE HAN LLP, 2400 GENG ROAD, SUITE 120, PALO ALTO, CA, 94303, US

(73) Assignee: Talus Medical, Inc., Los Gatos, CA (US)

(21) Appl. No.: 12/690,375

(22) Filed: Jan. 20, 2010

Related U.S. Application Data
(63) Continuation of application No. PCT/US2008/070441, filed on Jul. 18, 2008.
(60) Provisional application No. 60/951,120, filed on Jul. 20, 2007.

(51) Int. Cl. A61F 5/00; A61F 2/42

(52) U.S. Cl. 606/87; 623/21.18

(57) ABSTRACT

Methods and devices for deploying biological implants are disclosed. The biological implants can include orthopedic, multi-component ankle implants. The target site can be prepared by fixing a rigid, alignable guide or jig with saw holes to the bone(s). Saws configured to fit through the saw holes can then be inserted through the saw holes to cut the bone(s). The jig can then be removed. Slidable implants can be positioned. Implants needing to be forced into place can be attached to elongated members to gently hold the implant and to provide a non-implant surface on which to apply the force.

### CROSS-REFERENCE TO RELATED APPLICATION

This application is a continuation of PCT Application No. PCT/US2008/070441, filed 18 Jul. 2008, which claims the benefit of U.S. Provisional Application No. 60/951,120, filed 20 Jul. 2007, both of which are incorporated by reference herein in their entireties.

### FIELD OF THE INVENTION

This invention relates to methods and devices for deploying biological implants, more specifically for methods and devices for deploying bone implants.

### BACKGROUND OF THE INVENTION

FIGS. 1 and 2 illustrate anterior and lateral views of the tibia 6, talus 12 and fibula 2 (not shown in FIG. 2). A vertical axis 8 and an original talus thickness 10 are shown. The original talus thickness 10 is dependent on individual anatomical factors and the amount of pathological bone degradation. The talus has a talus head (caput tali) and talus neck (collum tali). The talus head has a rounded talus head crown.

Osteoarthritis or trauma can result in ankle pathology of uneven wear on, and/or direct trauma to, the surface of the talus. This commonly leads to cartilage erosion and subsequent break down of subchondral bone. Osteoarthritis and certain trauma on the talus are often treated by fusing the talus to the tibia. This fusion procedure results in loss of mobility of the ankle, and the expected complications resulting from a loss of mobility including gait changes, further stress-related injuries, and a reduction of the patient's overall mobility.

A secondary treatment for osteoarthritis in the talus—and in other bones—is to replace part of the damaged bone with a partial bone prosthesis. The partial bone prostheses, such as those for the talus or the long bones (e.g., femur, tibia, humerus, ulna), typically anchor into the remainder of the bone.

Implantation of prosthetic orthopedic implants is often accomplished by removing bone surrounding the implant site in order to provide the proper geometry to seat the implant. The implant is then positioned into place. A surgeon may have to make multiple passes with a straight saw or osteotome to remove the proper portion of bone. The osteotome position may also need to be altered between the cuts. Multiple cuts with no guide or limited guides can result in variable results from procedure to procedure.

Osteotome guides are known in the art, but are typically moved to accommodate various passes of a straight osteotome to accomplish anything other than a single straight removal of bone.

An osteotome is desired that can perform a single cut with multiple angles is desired. Furthermore, a guide for such an osteotome and methods of using both are desired.

### SUMMARY OF THE INVENTION

Methods and devices for deploying biological implants are disclosed. The biological implants can include two-piece or three-piece ankle implants. For example, the implants can have a prosthesis talus component and/or a prosthesis tibia component and/or a prosthesis floating component configured to be placed between the prosthesis talus component and the prosthesis tibia component. A guide can be used to prepare the target implantation site before the prosthesis components are implanted. One or more osteotomes can be used, for example directed by the guide, to cut target bone in preparation for implantation of the prosthesis components. An atraumatic holder or setting tool can be used to releasably hold, guide and move the prosthesis components during implantation.

The guide can be aligned at the target site. For example, a laser alignment line, or gravitation plumb bob, or anchored rod, or other alignment device can be secured to the lower leg (e.g., the tibia or patella) to provide a constant and reliable alignment line. The guide can have two or more holes for alignment pins. The guide can be aligned to the alignment line and fixed to the tibia or other bone, for example by inserting pins through the holes for the alignment pins, and fixing the pins into the bone. The alignment pins can be inserted through holes in a single plane (as shown in FIGS. 11a and 11b) or multiple planes.

The guide, also called a jig or frame, can be alignable with respect to the knee (e.g., patella) or tibia. The guide can have a rigid and fixed body. The guide body can be sufficiently thick, for example 19 mm (0.75 in.), for the material of the guide body, for example stainless steel, to prevent yaw, twist, or rotation of the guide during use (e.g., during cutting, for example to minimize cutting errors and tolerances). The guide body can have two or more slots passing therethrough to guide osteotomes. The slots can be at fixed positions with respect to each other in the guide. The guide can have a tongue or guide handle extending from the guide body at a talar declination angle, for example, to provide a field of view of the operating site for the surgeon during use.

A prosthesis holder or setting tool can be used to atraumatically and releasably hold the prosthesis talus component and/or the prosthesis tibia component and/or the prosthesis floating component. The prosthesis holder can be made in whole or part of soft material, such as polycarbonate, plastic, a soft rubberized material, or combinations thereof. The prosthesis holder can have an abutment away from the prosthesis to receive an impact force from a mallet or hammer. The prosthesis holder can then atraumatically deliver the impact force to the prosthesis component being held. The prosthesis holder can be long enough to extend out of the surgical field to allow a hammer or mallet to impact the abutment and to control work spaces far enough away from surgical field so the patient will not obstruct manipulation and use of the prosthesis holder.

The talus, tibia or floating components can also be positioned without use of the prosthesis holder, for example by positioning and inserting directly by hand.

One or more osteotomes (or saws or cutting tools) can be used to prepare the bones (e.g., tibia and talus) to receive the prosthesis components. The osteotomes can be configured to fit the slots in the guide. The osteotomes can have straight and/or rounded transverse cross-sections.

The osteotomes can have a cross-member. A leg can extend at an angle from either or both ends of the cross-member. The legs and cross-member can have a contiguous cutting edge. The osteotome can have a cutting edge with two, three or more contiguous elongated edge lengths (e.g., at the leading edge of the cross-member and legs). Each edge length can extend at an angle from the adjacent edge lengths. For example, a first cutting edge length along the cross-member can join at an angle with the second cutting edge length along a leg extending from the cross-member.

The osteotomes and guides can provide repeatable cuts with low tolerances. The cuts can match the fit needed for the prosthesis components.

Once the guide is fixed to the tibia and talus, the osteotomes can be inserted through the slots in the guide and cut the tibia and talus. The osteotomes and guides can be configured to preserve as much talus bone as possible, for example through the center of the talus head, while still sufficiently preparing the talus to receive the prosthesis. For example, the osteotomes can remove from about 3.18 mm (0.125 in.) or less to about 13 mm (0.5 in.) or less, for example about 6.4 mm (0.25 in.) or less of height of bone from the crown of the talus head. This height of removed bone can be substantially equivalent to the height of the shoulders of the prosthesis talus component.

The prosthesis components can then be positioned and fixed on the tibia and talus, for example with the prosthesis holder.

Once the prosthesis talus component and prosthesis tibia component have been fixed, the prosthesis floating component can be inserted between the prosthesis talus component and prosthesis tibia component, for example when surgically open joint is distended.

During the procedure, halo stabilizers can be fixed to (e.g., fixation screws can be drilled into) the bones. The halo stabilizers can be used to fix the talar angle with respect to the tibia, for example, to minimize error of placement of the prosthesis components.

### SUMMARY OF THE FIGURES

FIG. 1 is not the invention and illustrates an anterior view of the bones of the upper ankle.

FIG. 2 is not the invention and illustrates a lateral view of FIG. 1 sans fibula.

FIGS. 3a, 3b and 3d are front perspective, top perspective and side views, respectively, of a variation of the osteotomy guide.

FIGS. 3c′ and 3c″ are front views of variations of the osteotomy guide.

FIGS. 4a, 4b and 4c are front perspective, top, front and side views, respectively, of a variation of the osteotome.

FIG. 4d illustrates a variation of cross-section A-A of FIGS. 4a and 4c.

FIGS. 5 through 7″ are perspective views of variations of the prosthesis talus component.

FIGS. 8a′, 8b′, and 8c′ are front, bottom and side views, respectively, of the prosthesis talus component of FIG. 7′.

FIGS. 8a″ and 8b″ are front and bottom views, respectively, of the prosthesis talus component of FIG. 7″.

FIGS. 9a, 9b, and 9c are perspective, top, and side views of a variation of the prosthesis floating component.

FIG. 9d is a variation of section C-C of FIG. 9c.

FIGS. 10a, 10b and 10c are perspective, front and side views, respectively, of a variation of the prosthesis tibia component.

FIGS. 11a and 11b are anterior and lateral views, respectively, of a variation of a method for aligning and attaching the osteotomy guide to the tibia and talus.

FIGS. 12a and 12b are anterior and lateral views, respectively, of a variation of the osteotomy guide aligned and attached to the tibia and talus.

FIGS. 13a and 13b are anterior and lateral views, respectively, of the osteotomes aligned with and inserted into the osteotomy guide before beginning the osteotomy.

FIGS. 14a and 14b are anterior and lateral views, respectively, of the osteotomes concluding the cut of the osteotomy of the tibia and the talus.

FIGS. 15a and 15b are anterior and lateral views, respectively, of a variation of the ankle after the cut of the osteotomes and removal of the loose bone.

FIGS. 16a and 16b are anterior and lateral views, respectively, of a variation of the ankle after the cut of the osteotomes and removal of the loose bone.

FIGS. 17a and 17b are anterior and lateral views, respectively, of a method for implanting the prosthesis talus component with a removable prosthesis handle (not shown in FIG. 17a for illustrative purposes).

FIGS. 18a through 18c illustrate variations of prosthesis handles attached to a variation of the prosthesis talus component.

FIGS. 19a and 19b are anterior and lateral views, respectively, of the ankle with the prosthesis talus component in an implanted configuration.

FIGS. 20a and 20b are anterior and lateral views, respectively, of the ankle of FIGS. 19a and 19b with a variation of the tibia bone prosthesis attached.

FIGS. 21a and 21b are anterior and lateral views, respectively, of the ankle of FIGS. 19a and 19b with a variation of the tibia bone prosthesis attached.

FIGS. 22a and 22b are anterior and lateral views, respectively, of the ankle of FIGS. 19a and 19b with a variation of the tibia bone prosthesis attached.

FIGS. 23a and 23b are anterior and lateral views, respectively, of the ankle of FIGS. 19a and 19h with a variation of the tibia bone prosthesis attached.

FIGS. 24a and 24b are anterior and lateral views, respectively, of the ankle of FIGS. 20a and 20b with a variation of a prosthesis floating component.

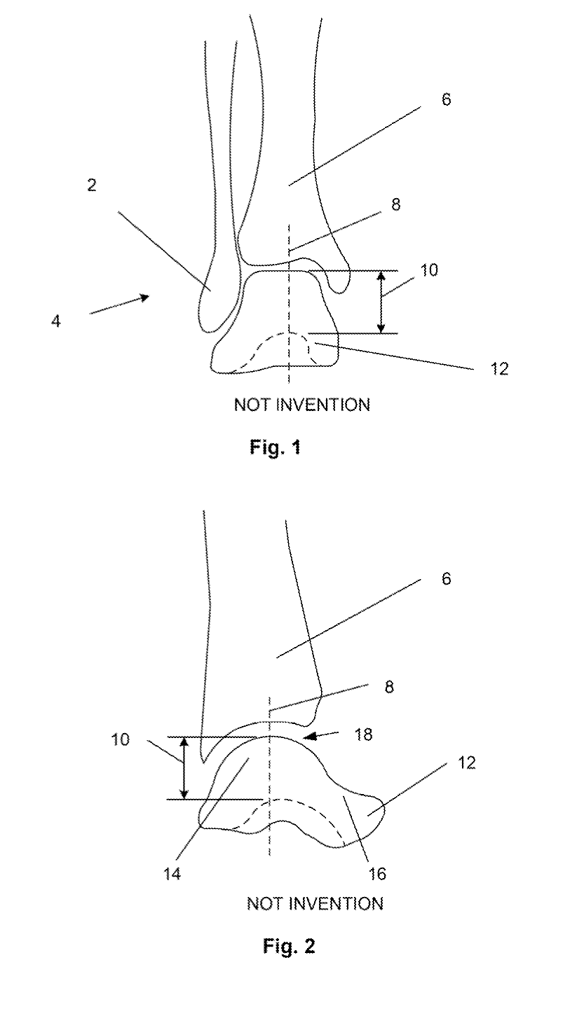

### DETAILED DESCRIPTION

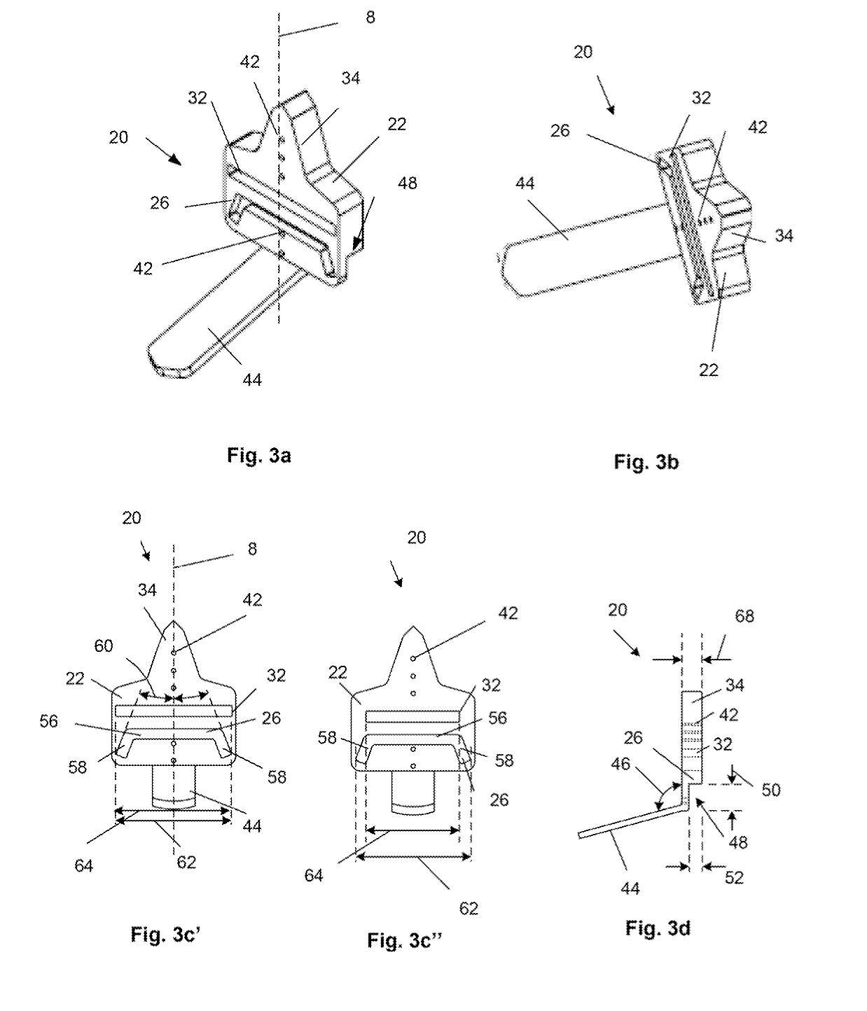

FIGS. 3a, 3b and 3d illustrate an osteotomy guide 20 that can have a guide body 22 having a guide body thickness 68. A talus port or slot 26 and/or tibia port or slot 32 can pass through the entire guide body thickness 68. The talus and tibia slots 26, 32 can be configured to receive and direct one or more osteotomes. The guide body 22 can have a narrowing guide neck 34 at the superior end of the guide body 22. The guide body 22 can have one, two or more alignment holes 42 passing through the entire guide body thickness 68. The alignment holes 42 can be configured in one or more lines, for example along a horizontally-centered, vertical axis 8. A superior end of the guide body 22 can narrow along the vertical axis 8 into a guide neck 34. The guide neck 34 can have additional alignment holes 42.

The guide body thickness 68 can be from about 6.4 mm (0.25 in.) to about 38 mm (1.5 in.), for example about 19 mm (0.75 in.). The guide body 22 can be sufficiently thick to prevent deformation of the guide body 22 during use, for example while fixed to adjacent, articulating bones.

The guide handle 44 can extend in an anterior direction from the guide body 22. The guide handle 44 can form a guide handle angle 46 with the plane of the guide body 22. The guide handle angle 46 can be from about 60° to about 150°, for example about 105°. The guide handle 44 can be integral with, or removably or fixedly attached to, the guide body 22. The guide handle 44 can have an elongated, substantially flat configuration. The guide handle 44 can be substantially rigid or flexible.

The guide body 22 can have a talus notch 48, for example, configured to avoid physical interference with the talus 12 during use. The talus notch 48 can have a talus notch height 50 and a talus notch depth 52. The talus notch height 50 can be from about 0 mm (0 in.) to about 25 mm (1.0 in.), for example about 13 mm (0.50 in.). The talus notch depth 52 can be from about 0 mm (0 in.) to about 13 mm (0.50 in.), for example about 6.4 mm (0.25 in.).

The guide body 22 can have a tibia slot and/or a talus slot 26, 32. The tibia slot 32 and the talus slot 26 can extend through the entire guide body 22. The tibia slot 32 can be a substantially straight or curved configuration. The talus slot 26 can have a talus slot body 56 having a substantially straight or curved configuration. The talus slot 26 can have a talus slot leg 58 extending contiguously (as shown) or separately from one or both ends of the talus slot body 56. The talus slot legs 58 can have substantially straight or curved configurations. The talus slot leg 58 can extend from the talus slot body 56 at a talus slot angle 60 with respect to the vertical axis 8. The talus slot angle 60 can be from about 0° to about 90°, more narrowly from about 20° to about 70°, for example about 40°.

FIG. 3c′ illustrates that the talus slot 26 can have a talus slot width 62. The tibia slot 32 can have a tibia slot width 64. The talus slot width 62 can be substantially equal to the tibia slot width 64. FIG. 3c″ illustrates that tibia slot width 64 can be smaller than the talus slot width 62. For example, the tibia slot width 64 can be about the width of the talus slot body 56.

The tibia slot width 64 can be from about 13 mm (0.5 in.) to about 64 mm (2.5 in.), for example about 36 mm (1.4 in.), or for example about 43 mm (1.7 in.). The talus slot width 62 can be from about 13 mm (0.5 in.) to about 76 mm (3.0 in.), for example about 43 mm (1.7 in.).

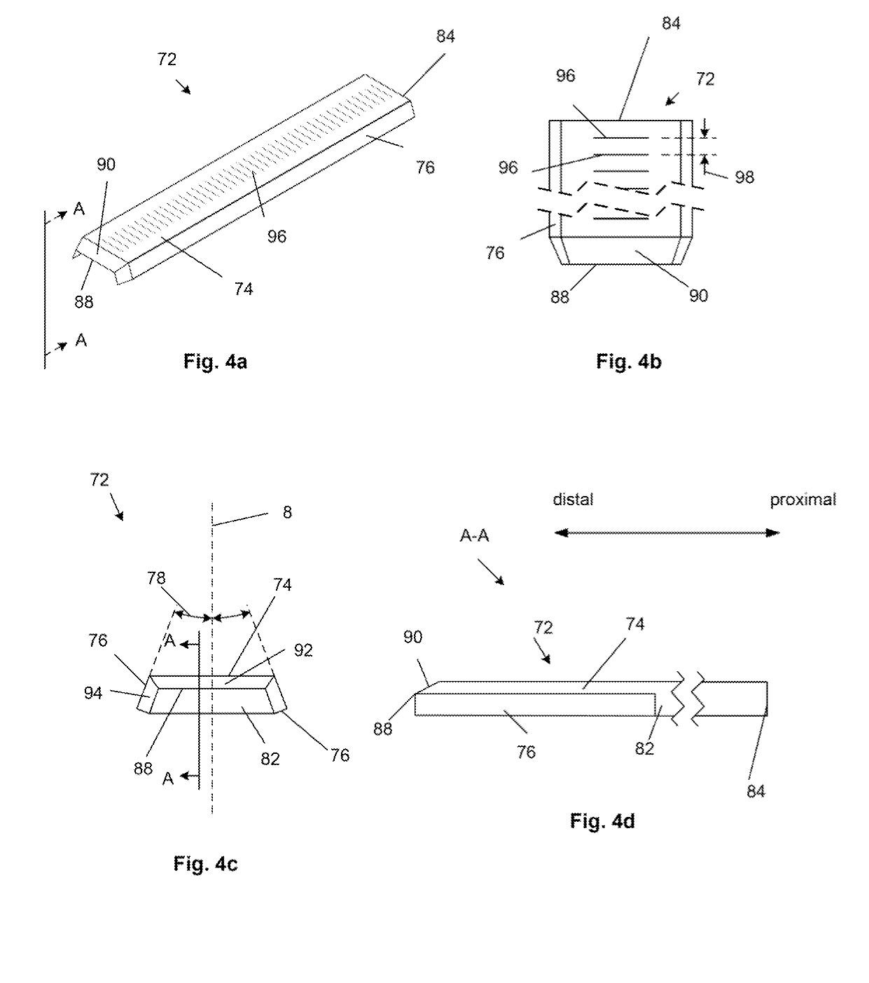

FIGS. 4a through 4d illustrate that a bone chisel, bone saw, or osteotome 72 (referred to herein as any of the above, particularly an osteotome), can have an osteotome roof. The osteotome cross-member 74 can have a substantially straight or curved configuration. The osteotome cross-member 74 can have an osteotome leg 76 extending contiguously from one or both sides of the osteotome cross-member 74. The osteotome legs 76 can have substantially straight or curved configurations. The osteotome leg 76 can extend from the osteotome cross-member 74 at an osteotome angle 78 with respect to the vertical axis 8. The osteotome angle 78 can be from about 0° to about 90°, more narrowly from about 20° to about 70°, for example about 40°. The osteotome angle 78 can be substantially equivalent to the talus slot angle 60. The osteotome 72 can be configured so part or all of the osteotome 72 can slidably fit through the talus and/or tibia slot 26, 32.

The proximal end of the osteotome 72 can have an osteotome body 82. When viewed from a longitudinal end of the osteotome 72, as shown in FIG. 4c, the osteotome body 82 can have the outer dimensions of the osteotome cross-member 74 and the osteotome legs 76, and can also be solid in the area defined by the hollow between the osteotome legs 76.

The proximal end of the osteotome 72 can be an osteotome butt 84, for example configured to receive a driving tool such as a hammer or mallet. The osteotome butt 84 can be configured to be a flat face. The osteotome butt 84 can be the proximal end of the osteotome body 82.

The distal end of the osteotome 72 can terminate in a cutting edge 88. For example, the cutting edge 88 can extend along the distal terminal ends of the osteotome cross-member 74 and the osteotome legs 76.

The osteotome 72 can taper into the cutting edge 88 at a cutting slope 90. The cutting slope 90 can extend along the distal ends of the osteotome cross-member 74 and the osteotome legs 76. The body 82 can have a body cutting slope 92. The legs 76 can each have a leg cutting slope 94.

The outside surface of the osteotome 72 can have one or more depth marks 96 indicating the depth along the osteotome 72. The depth marks 96 can be referred to during use to determine how deep the osteotome 72 has been inserted into tissue. The depth marks 96 can each be a transverse mark that can optionally have a number, letter or symbol adjacent to marks, for example to indicate the depth of that depth mark 96. The depth marks 96 can be spaced longitudinally along the osteotome 72. Adjacent depth marks 96 can be separated by a depth mark spacing length 98. The depth mark spacing length 98 can be from about 2.5 mm (0.10 in.) to about 20 mm (0.79 in.), for example about 5.0 mm (0.20 in.).

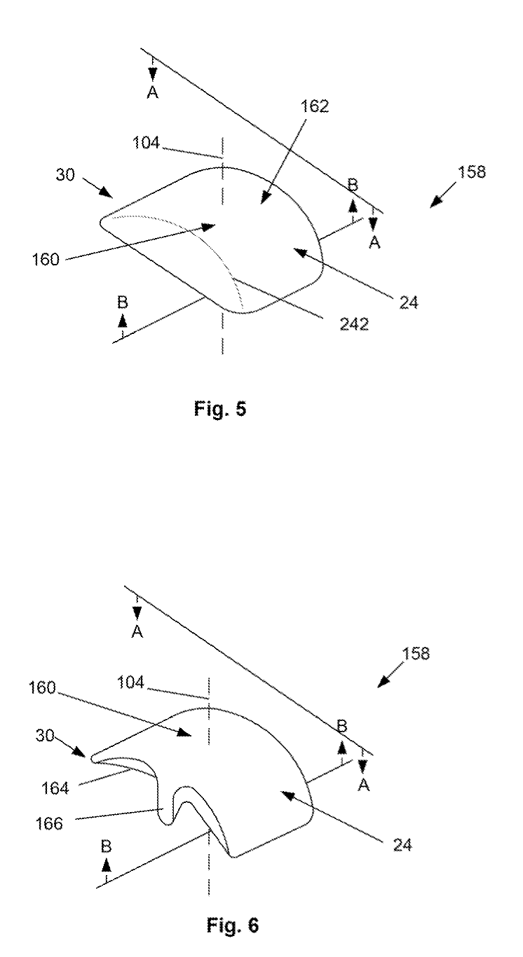

FIG. 5 illustrates that the partial bone prosthesis can have a prosthesis body 24. The contour line 242 shows curvature, such as an offset hemi-elliptical cam curvature, or hemi-oblong curvature, on the surface of the prosthesis body 24. The prosthesis body 24 can have a central axis 104. During use in a long bone, the central axis 104 can be substantially parallel and/or aligned with a longitudinal axis of the long bone. During use in the talus 12 or in a vertebra, the central axis 104 can be substantially parallel and/or aligned with a vertical axis 8.

The prosthesis body 24 can have a central portion 160. The central axis 104 can pass through the central portion 160. The prosthesis body 24 can have a perimeter anchor 30. The perimeter anchor 30 can be radially distal to the central axis 104. The perimeter anchor 30 can partially or completely surround the central portion 160.

The prosthesis can have a distal prosthesis surface 162. The distal prosthesis surface 162 can be configured to substantially match the exterior of the portion of the bone being replaced by the prosthesis. The proximal and distal prosthesis surfaces are proximal and distal, respectively, to the remainder of the bone which is being partially replaced.

FIG. 6 illustrates that the prosthesis body 24 can have one or more branches 166. The branches 166 can extend radially from the central axis. The branches 166 can extend substantially parallel, or not substantially parallel, to the central axis 104 at a radius from the central axis 104.

The prosthesis can have a proximal prosthesis surface 164. The proximal prosthesis surface 164 can be configured to attach to the bone.

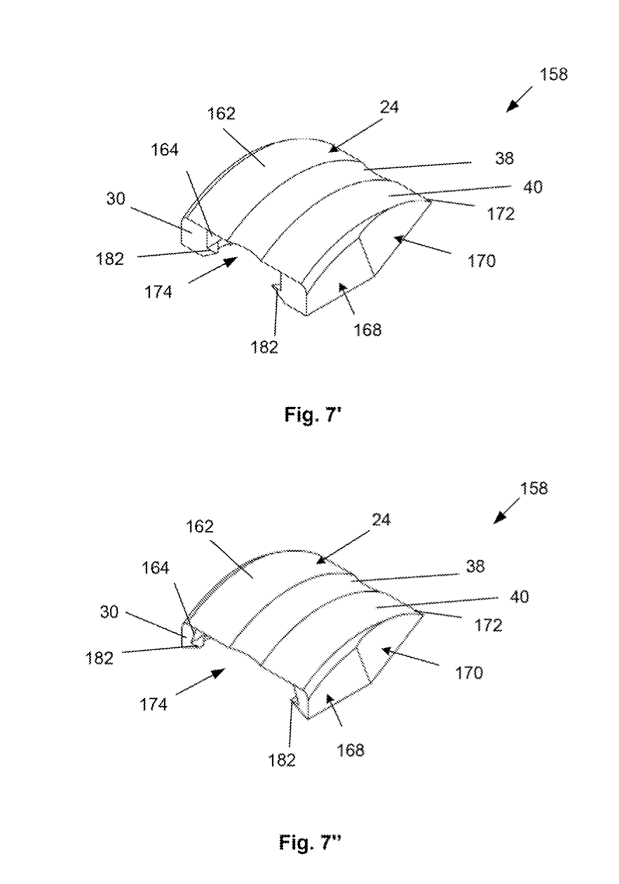

FIGS. 7′ and 7″ illustrate that the prosthesis body 24 can have one or more grooves 38 extending along a fore-aft (i.e., front-back or anterior-posterior) axis on the distal prosthesis surface 162. The groove 38 can be laterally centered on the prosthesis body 24. The groove 38 can be configured to align with a tongue in an adjacent implant or a protrusion in an adjacent bone to the groove 38. The groove 38 can be configured to minimize or otherwise restrict lateral movement of the implant with respect to the adjacent implant or adjacent bone to the groove 38.

The distal prosthesis surface 162 can have one or more shoulders 40 on each side of the groove and between grooves 38. The shoulders 40 can be flat and/or curved surfaces. The shoulders 40 and/or the grooves 38 can have low-friction coating, for example made from PTFE (e.g., Teflon® from E.I. du Pont de Nemours and Company of Wilmington, Del.).

The prosthesis body 24 can have a prosthesis flat 168 and a prosthesis rise 170. The prosthesis rise 170 can extend at an angle from the prosthesis flat 168 with measured parallel the up-down (i.e., dorsal-plantar or dorsal-palmar) axis.

The prosthesis body 24 can have a sharp edge 172 at the front and/or back of the prosthesis body 24. The prosthesis body 24 can have a flat, blunt face at the front and/or back of the prosthesis body 24.

The prosthesis body 24 can have a body channel. The bone channel 174 can pass through the prosthesis body 24 from the front to the back or from a first lateral side (i.e., left) to a second lateral side (i.e., right). The surface of the bone channel 174 can be formed by the proximal prosthesis surface 164. The perimeter anchor 30 can extend along two opposite sides of the bone channel 174. The perimeter anchor 30 can be vacant at the front port and/or back port of the bone channel 174.

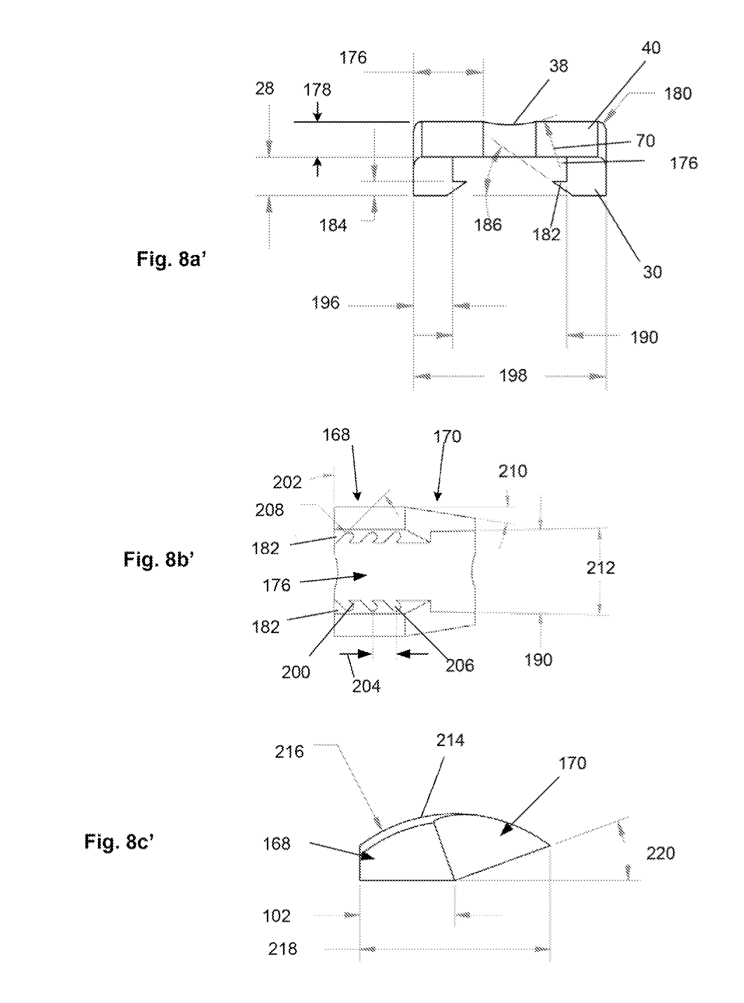

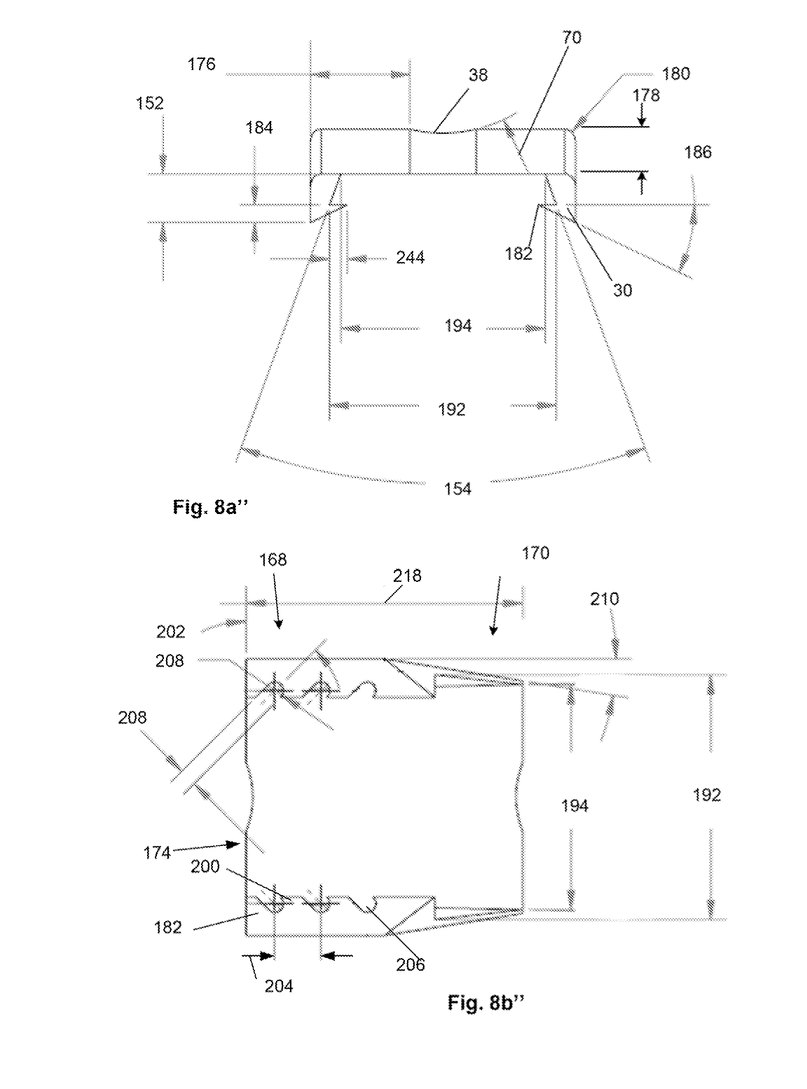

FIGS. 8a′ and 8a″ illustrates that the shoulders 40 can have shoulder widths 176. The shoulder width 176 can be from about 6.4 mm (0.25 in.) to about 19 mm (0.75 in.), for example about 12.7 mm (0.500 in.) or about 14.3 mm (0.563 in.). The shoulders 40 can have shoulder heights 178. The shoulder height 178 can be from about 3.18 mm (0.125 in.) to about 13 mm (0.5 in.), for example about 6.4 mm (0.25 in.) or about 10 mm (0.4 in.) or about 3.8 mm (0.15 in.).

The shoulders 40 can have a rounded transition to the sides of the prosthesis body having a distal chamfer radius 180. The distal chamfer radius 180 can be from about 0.08 mm (0.03 in.) to about 3.0 mm (0.12 in.), for example about 2 mm (0.06 in.).

The groove 38 can have a groove radius (of curvature) 70. The groove radius 70 can be from about 10 mm (0.4 in.) to about 41 mm (1.6 in.), for example about 20.7 mm (0.813 in.).

The ridge 182 can have a ridge height 184 and a ridge angle 186. The ridge height 184 can be from about 1.3 mm (0.05 in.) to about 13 min (0.5 in.), for example about 2.54 min (0.100 in.) or about 6.99 mm (0.275 in). The ridge angle 186 can be from about 15° to about 70°, for example about 35° or about 25.66°.

The bone channel 176 can have a bone channel width 190. The bone channel width 190 can be from about 10 mm (0.4 in.) to about 41 mm (1.6 in.), for example about 20.7 mm (0.813 in.).

As shown in FIG. 8a″, the bone channel 176 can vary in width from front to back and/or from top to bottom (i.e., distal to proximal). The bone channel 176 can have a maximum bone channel width 192 and a minimum bone channel width 194. The maximum bone channel width 192 can be from about 10 mm (0.4 in.) to about 41 mm (1.6 in.), for example about 32.61 mm (1.284 in.). The minimum bone channel width 194 can be from about 10 mm (0.4 in.) to about 41 mm (1.6 in.), for example about 29.36 mm 1.156 in.). A ridge width 244 can be the length from the ridge 182 to the radially inner surface of the remainder of the perimeter anchor 30 superior to the ridge 182.

The perimeter anchor 30 can have a perimeter anchor height 152 and a perimeter anchor width 196. The perimeter anchor height 152 can be from about 3.3 mm (0.13 in.) to about 16 mm (0.63 in.), more narrowly about from 3.3 mm (0.13 in.) to about 14 mm (0.55 in.), for example about 6.99 mm (0.275 in.), also for example about 9 mm (0.35 in.). The perimeter anchor width 196 can be from about 3.6 mm (0.14 in.) to about 14 mm (0.56 in.), for example about 7.14 min (0.281 in.).

The prosthesis body 24 can have a prosthesis body width 198 from about 17 mm (0.68 in.) to about 69.9 mm (2.75 in.), for example about 34.9 mm (1.375 in.), also for example about 38 mm (1.5 in.).

FIG. 8a″ illustrates that the bone channel 176 side of the perimeters can extend from the shoulders 40 at a perimeter extension angle 154. The perimeter extension angle 154 can be from about 0° to about 170′, more narrowly from about 15° to about 120°, for example about 40°.

FIGS. 8b′ and 8b″ illustrate that the ridge 182 can have one, two, three, four or more teeth 200. The teeth 200 can be sharpened. The teeth 200 can have a tooth angle 202 with respect to the face of closer end of the prosthesis body 24. The tooth angle 202 can be from about 20° to about 80°, for example about 45°. The teeth 200 can be separated from each other by a tooth gap 204. The tooth gap 204 can be from about 2 mm (0.08 in.) to about 12 mm (0.5 in.), for example about 3.96 mm (0.156 in.), also for example about 6.35 mm (0.250 in.). The teeth 200 can have a tooth slot 206 between the teeth. The tooth slot 206 can have a tooth slot diameter 208 from about 1 mm (0.05 in.) to about 5 mm (0.2 in.), for example about 2.4 mm (0.094 in.).

The sides of the prosthesis rise 170 can taper at a rise taper angle inward as it approaches the end of the prosthesis body 24. The rise taper angle 210 can be from about 0° to about 45°, more narrowly from about 4° to about 20°, for example about 9°.

The bone channel can taper at a bone channel angle 212. The bone channel angle 212 can be from about 0° to about 10°, for example about 2.4°.

FIG. 8c′ illustrates that the distal surface 214 can have a distal surface radius (of curvature) 216. The distal surface radius 216 can be from about 15 min (0.6 in.) to about 64 mm (2.5 in.), for example about 31.50 mm (1.240 in.).

The prosthesis flat 168 can have a prosthesis flat length 102. The prosthesis flat length 102 can be from about 8 mm (0.3 in.) to about 80 mm (3 in.), for example about 19.1 mm (0.750 in.). The prosthesis body 24 can have a prosthesis body length 218 from about 19 mm (0.75 in.) to about 80 mm (3 in.), for example about 38.10 mm (1.500 in.). The length of the prosthesis rise 170 can be the difference between the prosthesis flat length 102 and the prosthesis body length 218: about 0 mm (0 in.) to about 69 mm (2.7 in.), for example about 38 mm (1.5 in.).

The prosthesis rise 170 can have a rise lift angle 220 with respect to the bottom of the prosthesis flat 168. The rise lift angle 220 can be from about 0° to about 45°, more narrowly from about 10° to about 40°, for example about 20.2°.

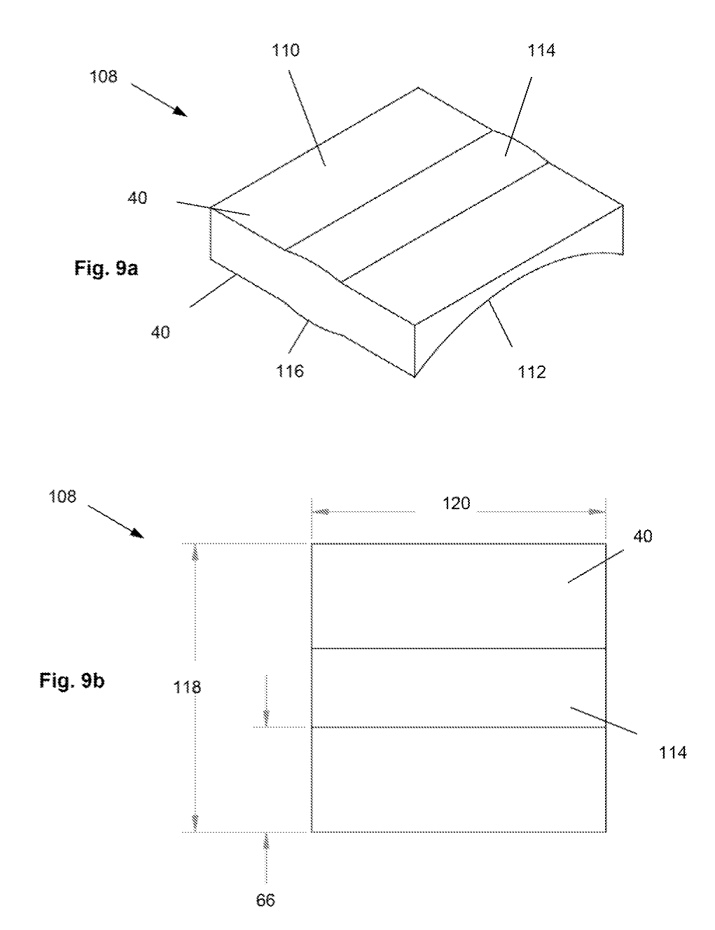

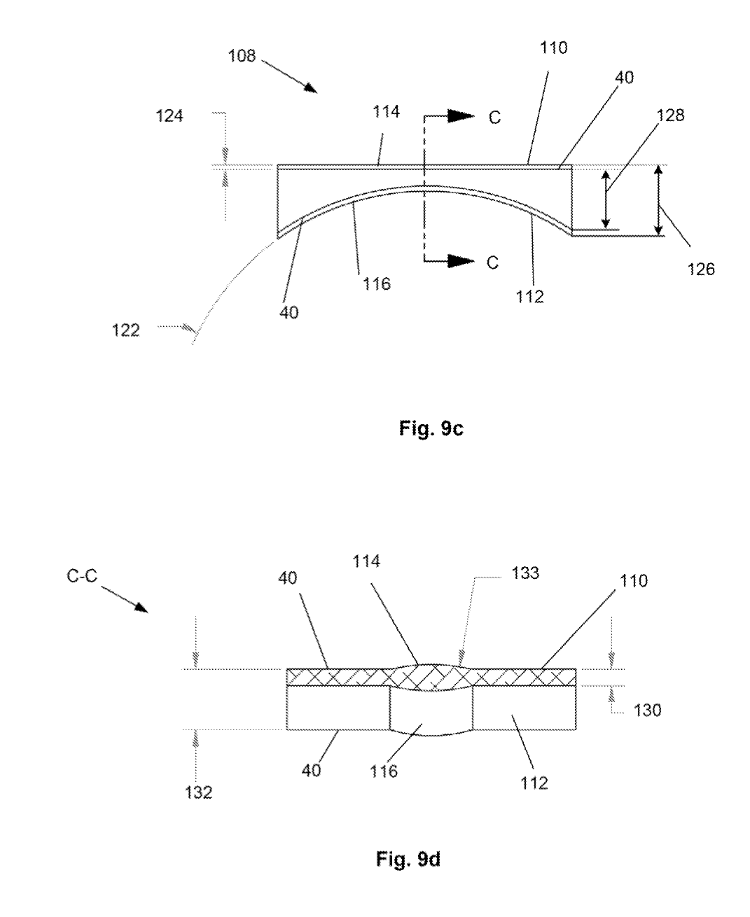

FIG. 9a illustrates that the prosthesis floating component 108 can have a substantially square or rectangular transverse section. The prosthesis floating section 108 can have a tibia-side surface 110 opposite of a talus-side surface 112. A tibia tongue 114 can extend from the tibia-side surface 110. The tibia tongue 114 can be configured to act as a slidable guide within the groove on the tibia prosthesis. A talus tongue 116 can extend from the talus-side surface 112. The talus tongue 116 can be configured to act as a slidable guide within the groove on the talus prosthesis.

FIG. 9b illustrates that the prosthesis floating component 108 can have a floating component width 118 and a floating component length 120. The floating component width 118 can be from about 18 mm (0.7 in.) to about 71 mm (2.8 in.), for example about 34.93 in. (1.375 in.). The floating component length 120 can be from about 18 mm (0.7 in.) to about 71 mm (2.8 in.), for example about 36 mm (1.4 in.).

The one or more shoulders 40 on the prosthesis floating component 108 can each have a shoulder width 66 from about 6.4 mm (0.25 in.) to about 0.25 mm 1.0 in.). The tibia and talus tongues 114, 116 can have the about same widths as the corresponding grooves 38 in the respective prosthesis components.

FIG. 9c illustrates that the shoulders 40 on the tibia-side surface 110 can be substantially flat. The talus tongue 116 and the shoulders 40 on the talus-side surface 112 can have a talus-side radius 122 (of curvature). The talus-side radius 122 can be from about 15 mm (0.6 in.) to about 64 mm (2.5 in.), for example about 32.13 mm (1.265 in.).

The talus-side surface 112 can be flat. The tibia-side surface 110 can be rounded.

The tongues 114, 116 can have a tongue height 124. The tongue height 124 can be from about 0.3 mm (0.01 in.) to about 1.3 mm (0.05 in.), for example about 5.6 mm (0.022 in.).

The floating component 108 can have a floating component height 126. The floating component height 126 without the tongues 114, 116 can be a tongueless height 128. The floating component height 126 can be from about 1.5 mm (0.06 in.) to about 17 mm (0.68 in.), for example about 8.43 mm (0.332 in.).

FIG. 9d illustrates that the prosthesis can have a minimum tongueless height 130, and a maximum tongueless height 132. The minimum tongueless height 130 can be about at the mid-point from front to back of the prosthesis floating component 108. The minimum tongueless height 130 can be from about 1 mm (0.04 in.) to about 4.1 mm (0.16 in.), for example about 2.0 mm (0.079 in.). The maximum tongueless height 132 can be about at the front and/or back ends of the prosthesis floating component 108. The maximum tongueless height 132 can be from about 3.6 mm (0.14 in.) to about 15 mm (0.58 in.), for example about 7.32 mm (0.288 in.).

The tongues 114, 116 can have the same or different tongue radii 133. The tongue radii 133 can be from about 10 mm (0.4 in.) to about 41 mm (1.6 in.), for example about 20.7 mm (0.813 in.). The tongue radii 133 can be about equal to the groove radii on the adjacent prosthesis component. For example, the groove radius 70 for the prosthesis tibia component 134 can be about the same as the tongue radius 133 for the tongue tibia-side surface 110 of the prosthesis floating component 108. The groove radius 70 for the prosthesis talus component can be about the same as the tongue radius 133 for the tongue talus-side surface 110 of the prosthesis floating component 108.

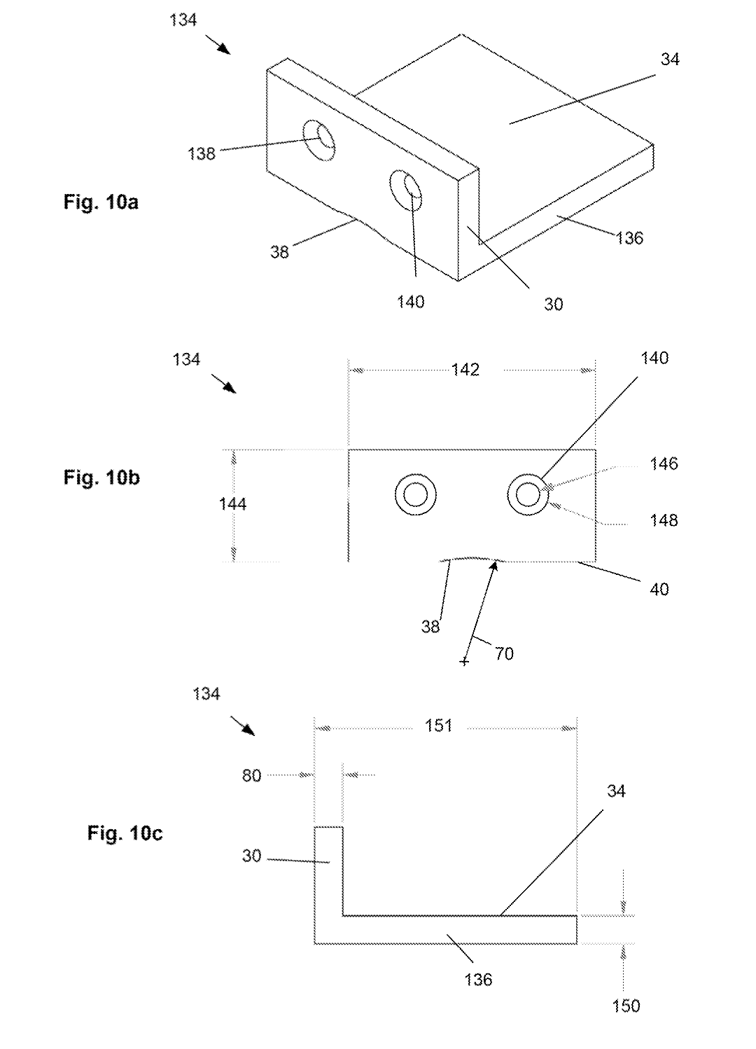

FIG. 10a illustrates a prosthesis tibia component 134 that can have a perimeter anchor 30 that extends from a base 136 along a single side of the base 136. The perimeter anchor 30 can extend at a right, obtuse, or acute angle from the base 136. The perimeter anchor 30 can extend from one, two, three, four or more sides of the base 136. The perimeter anchor 30 can have a first supplemental anchor port 138 and a second supplemental anchor port 140. The anchor ports 138, 140 can be straight or tapered. The anchor ports 138, 140 can be threaded or unthreaded. The prosthesis tibia component 134 can have a groove 38 configured to slidably engage the tibia tongue 114 on the prosthesis floating component 108. The prosthesis tibia component 134 can have a tongue 114, 116 configured to slidably engage the groove 38 on the prosthesis talus component 158, for example when in use without the prosthesis floating component 108.

The tongues 114, 116 on the prosthesis floating component 108 can either or both be grooves 38, and the grooves 38 on the prosthesis tibia component 134 and the prosthesis talus component 158 can either or both be tongues 114, 116 to engageably match the corresponding structure on the prosthesis floating component 108.

FIG. 10b illustrates that the prosthesis tibia component 134 can have a tibia component length 142 and a tibia component height 144. The tibia component length 142 can be from about 17 mm (0.65 in.) to about 69 mm (2.7 in.), for example about 34.93 mm (1.375 in.). The tibia component height 144 can be from about 7.9 mm (0.31 in.) to about 31.8 mm (1.25 in.), for example about 15.9 mm (0.625 in.).

The anchor ports 138, 140 can have an anchor port inner radius 146 and an anchor port outer radius 148, for example if the anchor port is tapered or threaded. The anchor port inner radius 146 can be from about 1.5 mm (0.06 in.) to about 3.3 mm (0.13 in.), for example about 1.7 mm (0.065 in.). The anchor port outer radius 148 can be from about 1.5 mm (0.06 in.) to about 5.8 mm (0.23 in.), for example about 2.87 mm (0.113 in.).

The groove radius 70 of the prosthesis tibia component 134 can be about equal to the groove radius 70 for the prosthesis talus component 158.

FIG. 10c illustrates that the perimeter anchor 30 can have a perimeter anchor width 196. The perimeter anchor width 196 can be from about 2 mm (0.07 in.) to about 8 mm (0.3 in.), for example about 3.81 mm (0.150 in.).

The base 136 can have a base height 150. The base height 150 can be from about 2 mm (0.07 in.) to about 8 mm (0.3 in.), for example about 3.81 mm (0.150 in.).

The prosthesis tibia component 134 can have a tibia component width 151. The tibia component width 151 can be from about 17 mm (0.7 in.) to about 71 mm (2.8 in.), for example about 35.56 mm (1.400 in.).

Any or all elements of the prosthesis and/or other devices or apparatuses described herein, including the prosthesis body 24 of the talus prosthesis, prosthesis floating component 108, and/or tibial prosthesis, or any other prosthesis, can have a surface finish to about 1.6 □m (63 □in.) or less.

Any or all elements of the prosthesis and/or other devices or apparatuses described herein, including the prosthesis body 24 of the talus prosthesis, prosthesis floating component 108, and/or tibial prosthesis, or any other prosthesis, can be made from, for example, a single or multiple stainless steel alloys, nickel titanium alloys (e.g., Nitinol), other titanium alloys, cobalt-chrome alloys ELGILOY® from Elgin Specialty Metals, Elgin, Ill.; CONICHROME® from Carpenter Metals Corp., Wyomissing, Pa.), aluminum and aluminum alloys (e.g., 6060-T6 aluminum, 6061-T6 aluminum), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET)/polyester (e.g., DACRON® from E. I. Du Pont de Nemours and Company, Wilmington, Del.), polypropylene, (PET), polytetrafluoroethylene (PTFE), expanded PTFE (ePTFE), polyether ether ketone (PEEK), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), polylactic acid (PLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, bone morphogenic protein (BMP), osteogenic powder, beads of bone) any of the other materials listed herein or combinations thereof. Examples of radiopaque materials are barium sulfate, zinc oxide, titanium, stainless steel, nickel-titanium alloys, tantalum and gold.

Any or all elements of the prosthesis and/or other devices or apparatuses described herein can be or have a matrix for cell ingrowth (e.g., as described supra) or used with a fabric, for example a covering (not shown) that acts as a matrix for cell ingrowth. The matrix and/or fabric can be, for example, polyester (e.g., DACRON® from E. I. Du Pont de Nemours and Company, Wilmington, Del.), polypropylene, PTFE, ePTFE, nylon, extruded collagen, a cobalt-chrome alloy matrix, silicone or combinations thereof.

The elements of the prosthesis and/or other devices or apparatuses described herein and/or the fabric can be filled and/or coated with an agent delivery matrix known to one having ordinary skill in the art and/or a therapeutic and/or diagnostic agent. The agents within these matrices can include radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Any or all parts of the prosthesis or other elements, tools, bones or other parts of the implant site can be coated with hydroxyapetite. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic. Aneurysm Wall and Its Effect on Inflammation and Proteolysis, Brit. J. Surgery 86 (6), 771-775; Xu et al, Sp1 Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

### Method of Use

Any of the variations of the devices, methods and elements thereof described in PCT Application No. PCT/US2007/063233 filed 2 Mar. 2007, which is incorporated by reference herein in its entirety, can be used herein.

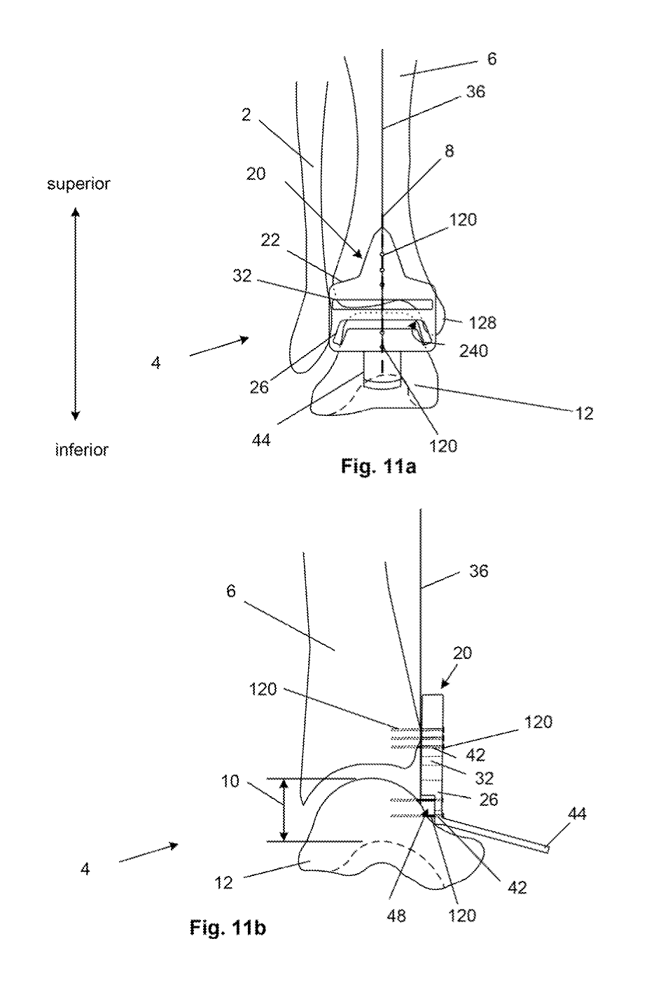

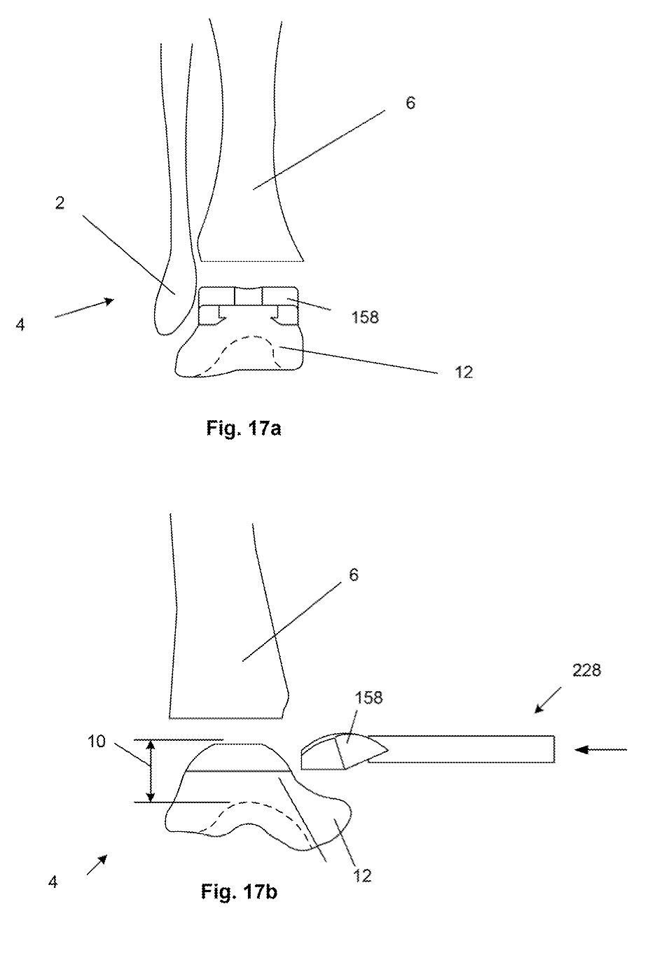

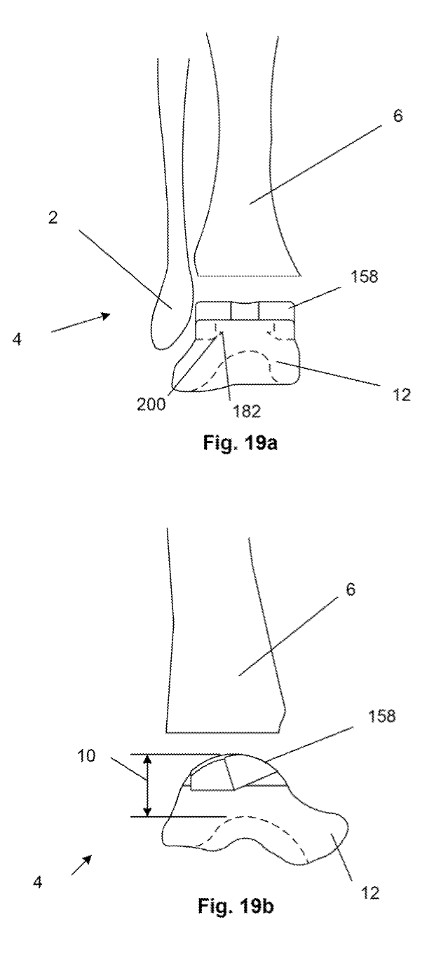

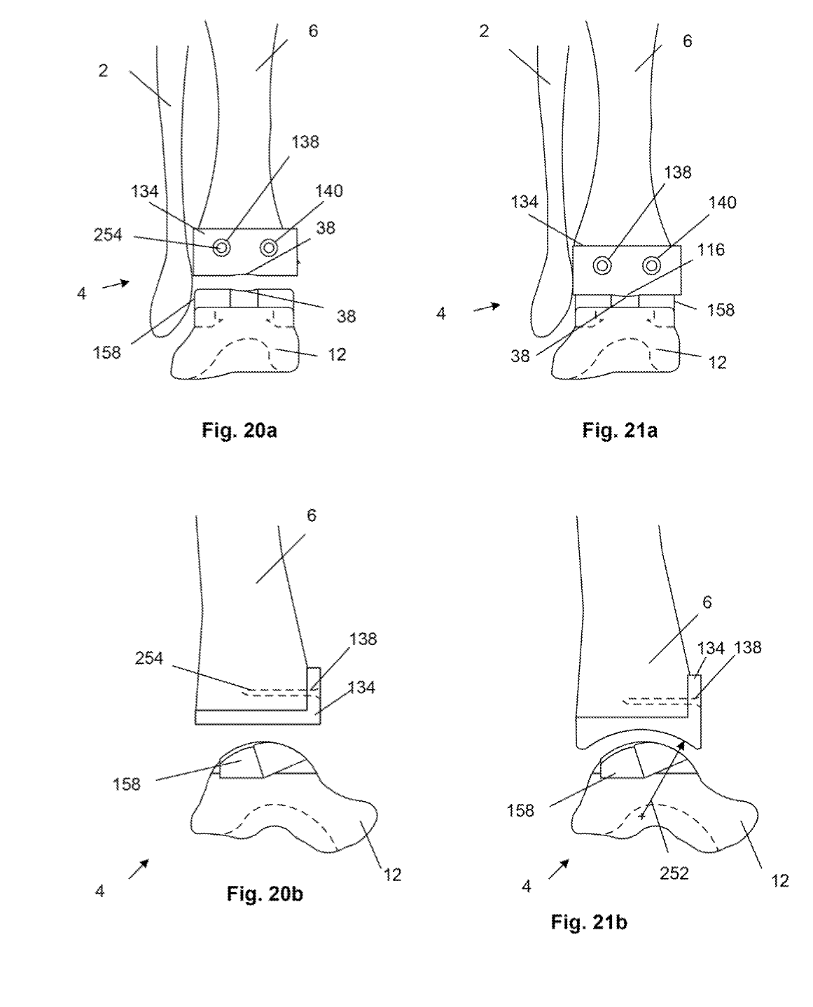

FIGS. 11a and 11b illustrate that an alignment line 36 can be positioned in front of the tibia 6. For example, the alignment line 36 can be a plumb bob lire attached to 11 and hanging from the patella or a laser line aligned with the patella. The guide can be positioned so the plane of the vertical axis of the guide aligns with the alignment line 36. The guide can be positioned so the guide is substantially against the tibia 6 and the talus 12. The guide can be positioned so the tibia slot 32 can align with the inferior end of the tibia 6 to match the size of the prosthesis tibia component (e.g., see FIGS. 20a-23b and infra). The guide can be positioned so the talus slot 26 can align with the superior end of the talus 12 to match the size of the prosthesis talus component (e.g., see FIGS. 17a-b, 19a-b and infra). The talus slot 26 and/or tibia slot 6 can be configured to overlap with some or all of the medial malleolus articular facet 240 during use.

With the guide in a desired position, attachment pins 120 can be inserted through the alignment holes 42. The attachment pins 120 can be inserted into the talus 12 and/or tibia 6, as shown. The attachment pins 120 can detachably fixedly attach the guide to the tibia 6 and/or talus 12. The attachment pins 120 can have heads with larger diameters than the alignment holes 42, for example, to prevent the attachment pin 120 from being deployed too deep into the tibia 6 and/or talus 12.

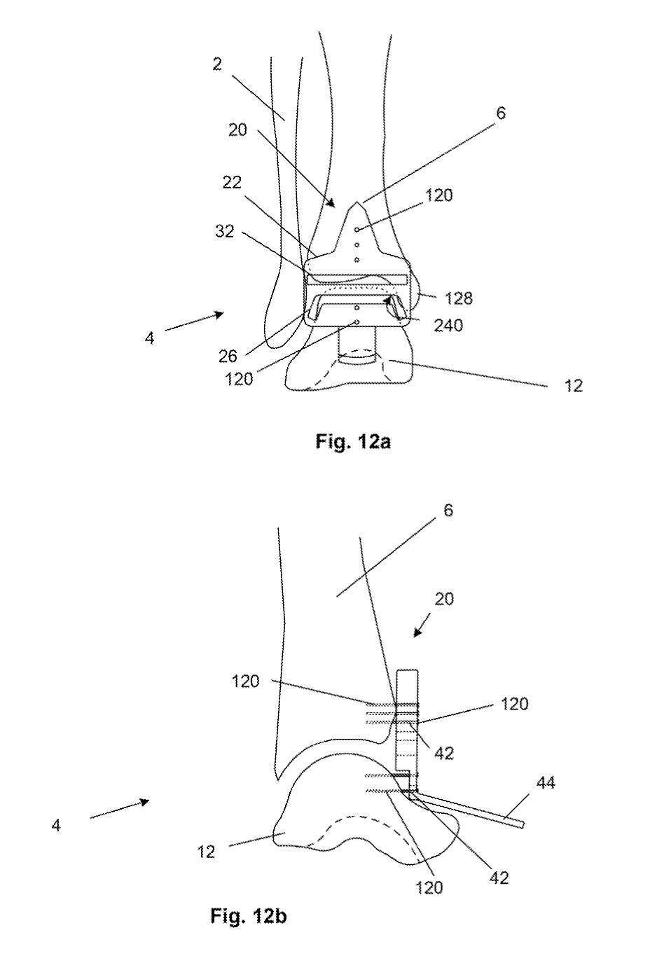

FIGS. 12a and 12b illustrate that the alignment line 42 can be removed once the guide is secured to the tibia 6 and talus 12, for example with the attachment pins 120.

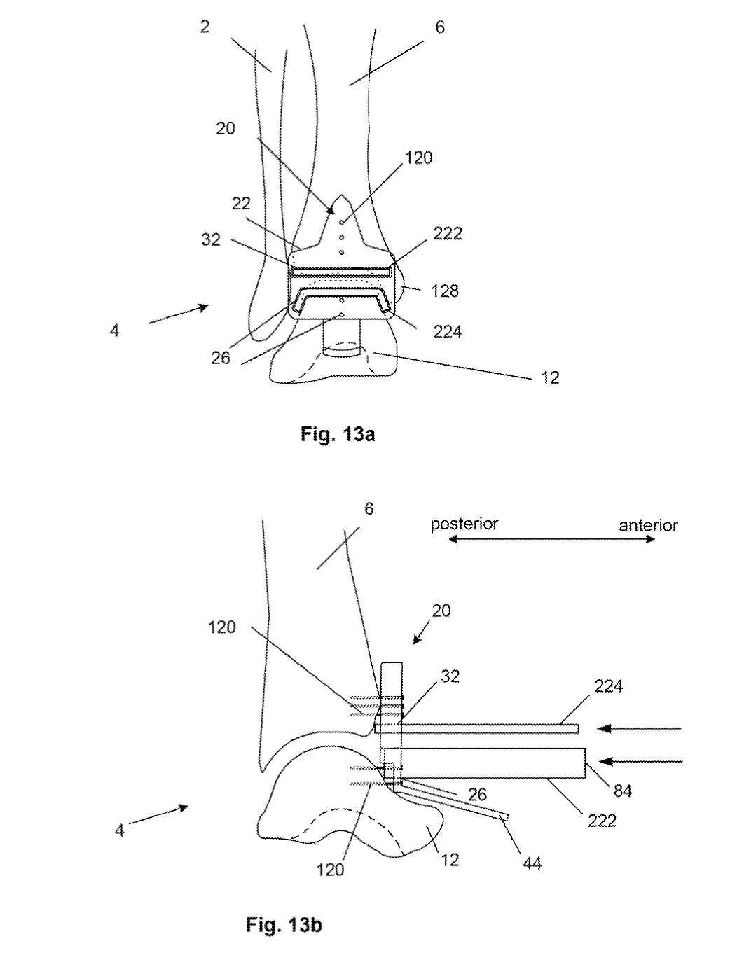

FIGS. 13a and 13b illustrate that a tibia osteotome 222 can be aligned with the tibia slot 32. A talus osteotome 224 can be aligned with the talus slot 26. The tibia osteotome 222 and/or the talus osteotome 224 can have substantially straight or curved transverse cross-sections or be as shown and described in FIGS. 4a-4d. The osteotomes 72 can be aligned and used subsequent to each other or concurrently. The osteotomes 72 can be driven posteriorly subsequently or concurrently, for example by impacting the osteotome butt with a force, as shown by arrows.

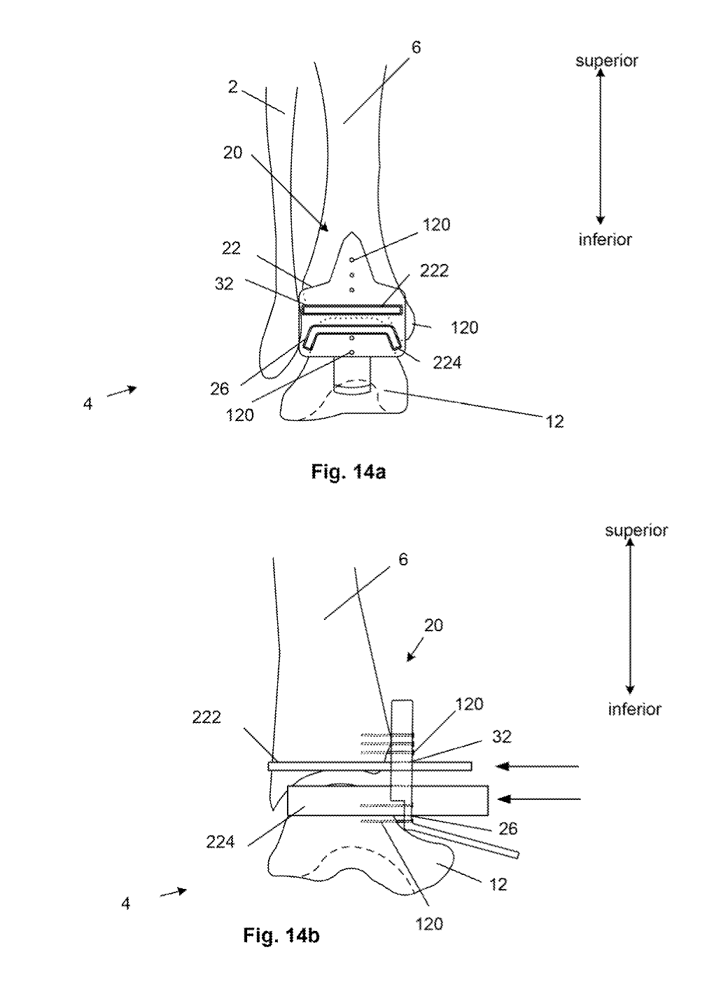

FIGS. 14a and 14b illustrate that the tibia osteotome 222 can be driven through the tibia 6. The tibia osteotome 222 can severe the medial malleolus 128 from the remainder of the tibia 6 or leave the medial malleolus 128 integral with the remainder of the tibia. The terminal inferior end of the tibia can be severed from the remainder of the tibia.

The talus osteotome 224 can be driven through part or all of the depth of the talus 12.

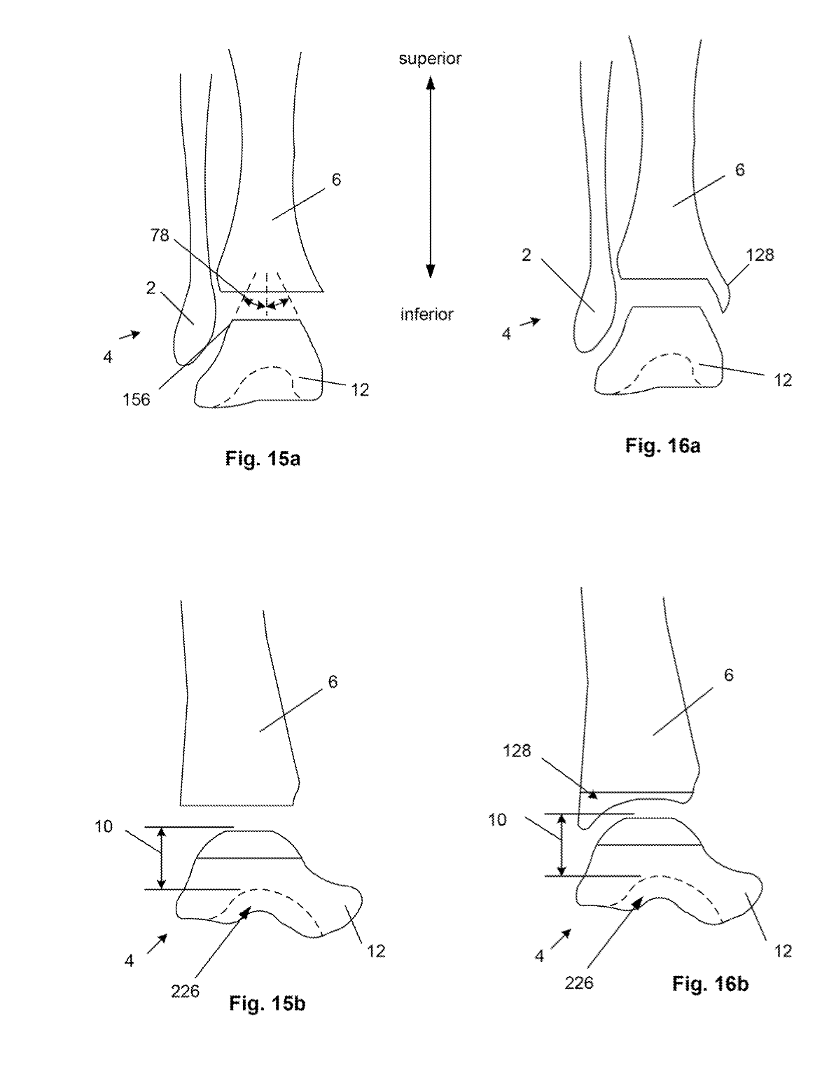

FIGS. 15a and 15b illustrate that the loose bone can be removed after the osteotomes 72 have cut the tibia 6 and/or the talus 12. The inferior end of the tibia 6 can be planed by the tibia osteotome 222. The inferior end of the tibia 6 can be configured to have a surface approximating an anatomical transverse plane. FIGS. 16a and 16b illustrate that the medial malleolus can be left intact and that the plane can be cut starting medially (with respect to the tibia, not the body) of the medial malleolus 128.

The superior end of the talus 12 can be planed by the talus osteotome 224. The talus osteotome 224 can also cut one or two side planes 156 part-way down the sides of the talus 12 starting from the superior end of the talus 12. The side planes 156 can extend from the superior end of the talus 12 at the osteotome angle 78.

The depth of bone cut from the talus 12 can leave a substantially large percentage (e.g., greater than about 50% or greater than 75%, or greater than 90%) of the original talus thickness 10 as measured near the center of the talus 12, for example at the sinus tarsi 226, as shown in FIGS. 15b and 16b.

FIGS. 17a and 17b illustrate that the prosthesis talus component 158 can be controllably releasably attached or otherwise removably attached to a prosthesis holder 228. The prosthesis talus component 158 can be aligned with the planes 156 cut in the talus 12. The prosthesis talus component 158 can be translated posteriorly, as shown by arrow. When the prosthesis talus component 158 initially contacts the talus 12, the ridge 182 and teeth 200 can interference fit against bone. A force can then be applied (e.g., an impact force, for example by striking the proximal end of the prosthesis holder with a hammer or mallet) to force the teeth 200 and ridge 182 through the bone.

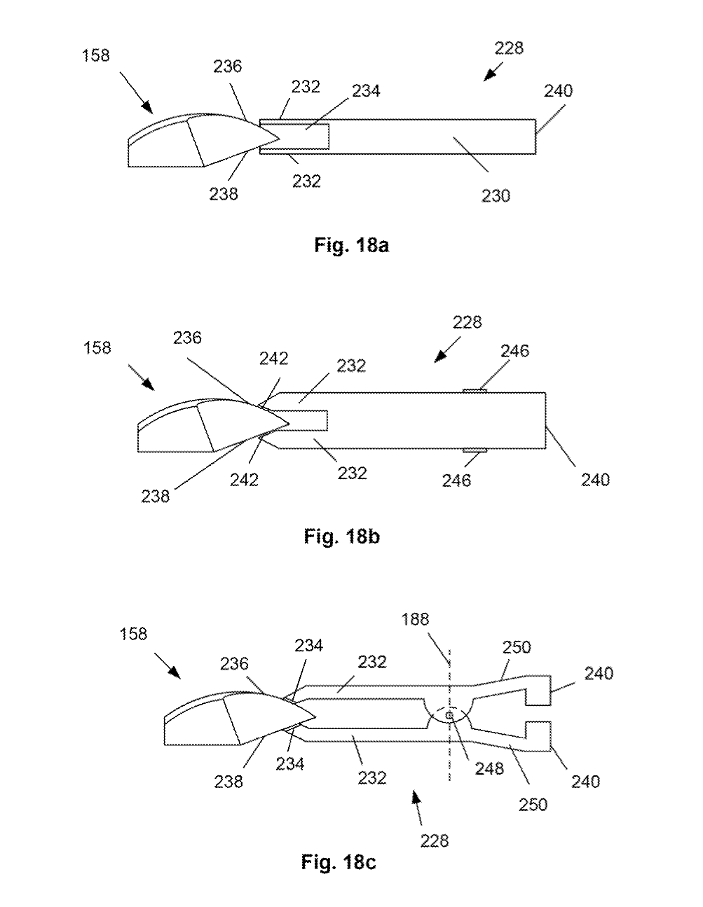

FIG. 18a illustrates a prosthesis holder 228 than can have a rigid holder body 230 and holder arms 232. A deformable or resilient holder pad 234 can be located between the holder arms 232 at the distal end of the prosthesis holder 228. The holder pad 234 can atraumatically fit the proximal top surface 236 and proximal bottom surface 238 of the prosthesis talus component 158. The holder pad 234 can be compressed by the prosthesis talus component 158 so that the holder pad 234 is between the prosthesis talus component 158 and the holder arms 232. The prosthesis talus component 158 can adhere to the prosthesis holder 228 by friction fit against the holder pad 234 and/or adhesive.

The prosthesis holder 228 can have a hammer abutment 240 at the proximal end of the prosthesis holder 228. The prosthesis holder 228 can be configured to receive an impact force from a hammer or mallet against the hammer abutment 240. The prosthesis holder 228 can be configured to transmit an impact force atraumatically to the prosthesis talus component 158.

FIG. 18b illustrates that the prosthesis holder can have an atraumatic retractable pad 242 at the end of each holder arm 232. The retractable pads 242 can be configured to friction fit against the proximal top surface 236 and proximal bottom surface 238 of the prosthesis talus component 158. The retractable pads 242 can be deployed by one or more spring-loaded mechanisms internal to the prosthesis holder 228. The prosthesis holder 228 can have one or more controls, such as buttons 246, that can controllably retract or extend the retractable pads 242 into the holder arms 232. Retracting the retractable pads 242 can detach the prosthesis talus component 158 from the prosthesis holder 228.

FIG. 18c illustrates that the holder arms 232 can be rotatably attached at a hinge 248. Each holder arm 232 can be integral with or fixedly attached to a holder leg 250. The proximal ends of the holder legs 250 can have hammer abutments 240. The distal ends of the holder arms can have atraumatic holder pads 234. The holder pad 234 on a first holder arm can be configured to atraumatically fit the proximal top surface 236 of the prosthesis talus component 158. The holder pad 234 on a second holder arm can be configured to atraumatically lit the proximal bottom surface 238 of the prosthesis talus component 158.

The distance between the center of each hammer abutment 240 and the hinge 248 when measured along the lever arm axis 188 can be larger than the distance between the hinge 248 and the center of the contact patch of each holder pad 234 against the prosthesis talus component 158 when also measured along the lever arm axis 188. For example, when an impact force is delivered to the hammer abutments 240, the impact force can increase the squeeze force of the holder arms 250 against the prosthesis talus component 158 (i.e., tighten the grip of the holder arms).

The holder pads 234 and retractable pads 242 can be coated, made entirely from, or made partially from a plastic, polycarbonate, plastic, rubber, a soft rubberized material, or other polymer, metal, ceramic, biomaterial such as bone (e.g., compressed morselized bone) or BMP, or combinations thereof. The holder pads 234 and retractable pads 242 can be soft enough to not scar the prosthesis talus component 158 while delivering impact force from a mallet or hammer impact on the hammer abutment 240.

FIGS. 19a and 19b illustrate that the prosthesis talus component 158 can be positioned on the talus 12. The ridge 182 and/or teeth 200 can be substantially embedded in the talus 12. The ridge 182 and/or teeth 200 can fix the prosthesis talus component 158 to the talus 12. Measured with the talus 12 and the prosthesis talus component 158, the original talus thickness 10 can be restored.

FIGS. 20a and 20b illustrate that the prosthesis tibia component 134 can be attached to the tibia 6, for example by inserting fixation pins 254 or screws through anchor ports 138, 140 and fixing the pins or screw into the tibia 6. The grooves 38 on the prosthesis tibia component 134 and the prosthesis talus component can be aligned horizontally. The prosthesis tibia component 134 can be positioned sufficiently superior on the tibia to allow for the prosthesis floating component 108 to be positioned between the prosthesis tibia component 134 and the prosthesis talus component 158.

FIGS. 21a and 21b illustrate that the prosthesis tibia component 134 can be positioned to slidably contact the prosthesis talus component 158. The prosthesis floating component 108 can be absent. The prosthesis tibia component 134 can have a talus tongue 116. The talus tongue 116 can be configured to slidably fit in the groove 38 of the prosthesis talus component 158.

The distance between the tibia slot 32 and the talus slot on the guide can be configured based on whether a prosthesis floating component 108 is to be inserted between the prosthesis tibia component 134 and the prosthesis talus component.

The prosthesis tibia component 134 can have an inferior surface radius of curvature 252, for example that is substantially equivalent to the radius of curvature of the superior surface of the prosthesis talus component 158.

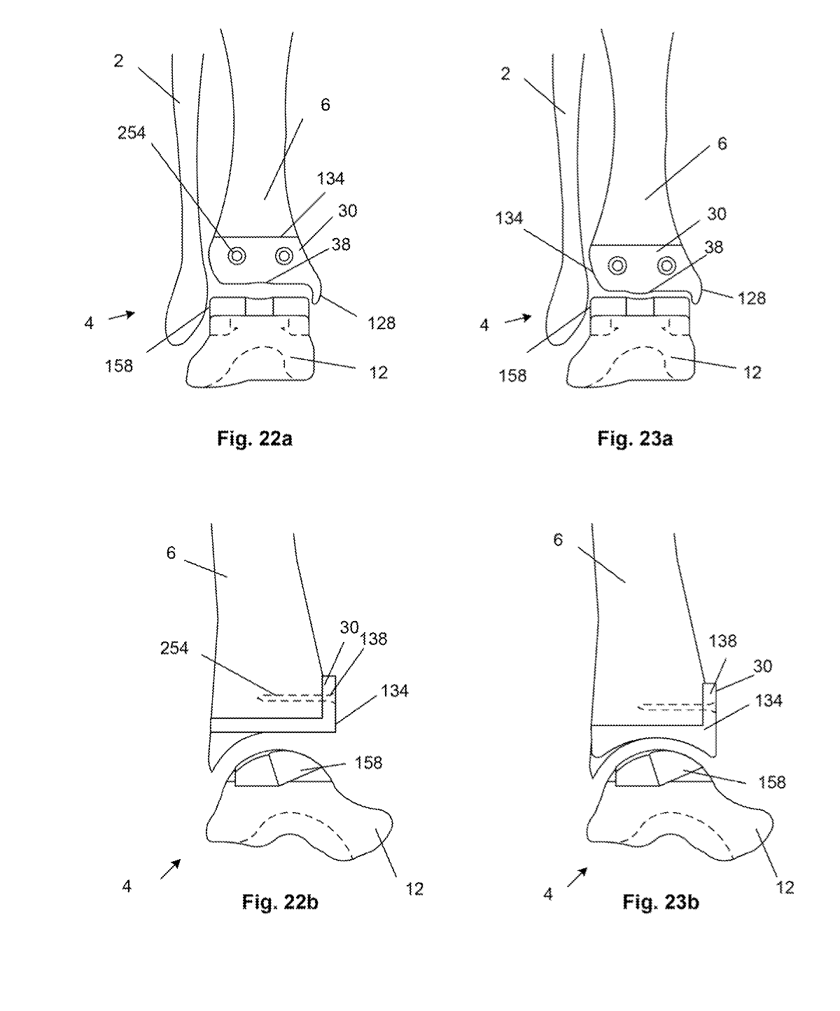

FIGS. 20a and 20b illustrate that the medial malleolus 128 can be removed. FIGS. 22a and 22b illustrate a variation of the prosthesis and method of FIGS. 20a and 20b where the medial malleolus 128 can be left attached to the tibia 6 or that the tibia prosthesis component 134 can have a configuration to approximate the medial malleolus 128. Further, the perimeter anchor 30 can be configured to approximate the shape of the tibia 6.

FIGS. 21a and 21b illustrate that the medial malleolus 128 can be removed. FIGS. 23a and 23h illustrate a variation of the prosthesis and method of FIGS. 21a and 21b where the medial malleolus 128 can be left attached to the tibia 6 or that the tibia prosthesis component 134 can have a configuration to approximate the medial malleolus 128. Further, the perimeter anchor 30 can be configured to approximate the shape of the tibia 6.

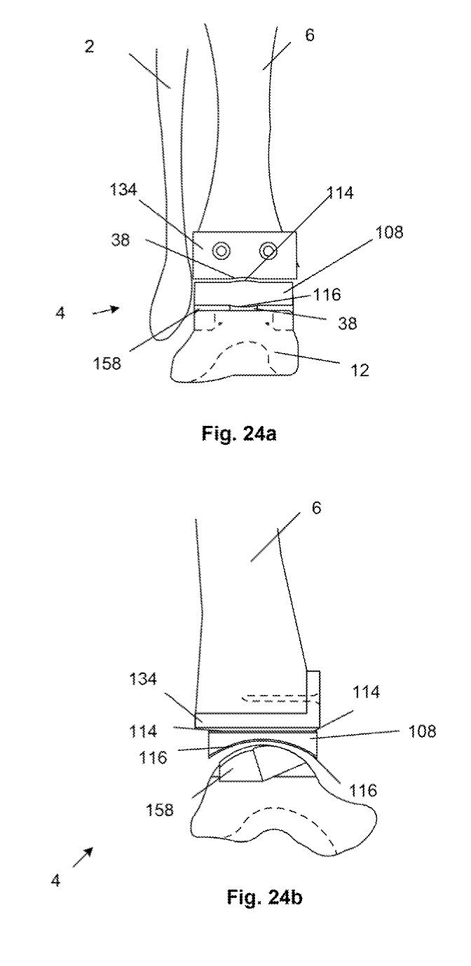

FIGS. 24a and 24b illustrate that the prosthesis floating component 108 can be inserted between the prosthesis tibia component 134 and the prosthesis talus component 158. The prosthesis floating component 108 can be configured to slidably contact the prosthesis talus component and the prosthesis tibia component 134. The tibia tongue 116 can slidably fit in the groove 38 in the prosthesis tibia component 134. The talus tongue 116 can slidably fit in the groove 38 in the prosthesis talus component 158. The prosthesis floating component 108, the prosthesis tibia component 134, and the prosthesis talus component 158 can be made from the same and/or different materials.

It is apparent to one skilled in the art that various changes and modifications can be made to this disclosure, and equivalents employed, without departing from the spirit and scope of the invention. Elements shown with any variation are exemplary for the specific variation and can be used in combination with, or otherwise on or in, other variations within this disclosure.

We claim:

1. An osteotomy guide comprising:
a guide body,
wherein the guide body has a first elongated port extending substantially through the entire thickness of the guide body, and wherein the guide boy has a second elongated port extending substantially through the entire thickness of the guide body, and wherein the second elongated port extends at a first angle from the first elongated port.

2. The guide of claim 1, wherein the second elongated port extends from a first end of the first elongated port.

3. The guide of claim 2, wherein the guide body has a third elongated port extending substantially through the entire thickness of the guide body, wherein the third elongated port extends at a second angle from the first elongated port, wherein the third elongated port extends from a second end of the first elongated port.

4. The guide of claim 1, wherein the first elongated port is integral with the second elongated port.

5. The guide of claim 4, wherein the guide body has a first elongated port extending substantially through the entire thickness of the guide body, wherein the third elongated port extends at a second angle from the first elongated port, wherein the first elongated port is integral with the second elongated port.

6. The guide of claim 1, wherein the first angle is from about 5° to about 175°.

7. The guide of claim 6, wherein the guide body has a first elongated port extending substantially through the entire thickness of the guide body, wherein the third elongated port extends at a second angle from the first elongated port, wherein the second angle is from about 5° to about 175°.

8. The guide of claim 1, wherein the first angle is from about 20° to about 70°.

9. The guide of claim 1, wherein the guide body has a guide body thickness greater than about 0.375 in.

10. The guide of claim 1, wherein the guide body has a first elongated port extending substantially through the entire thickness of the guide body, wherein the third cutting tool elongated port extends at a second angle from the first cutting tool elongated port.

11. The guide of claim 1, wherein the guide body has a fourth elongated port.

12. The guide of claim 11, wherein the fourth elongated port is not integral with the first elongated port.

13. The guide of claim 11, wherein the fourth elongated port is parallel with the first elongated port.

14. An osteotomy device comprising:
an osteotome body having a cutting edge, wherein the cutting edge comprises a first elongated edge and a second elongated edge, and wherein the second elongated edge extends from the first elongated edge at first angle.

15. The device of claim 14, wherein the first angle is from about 5° to about 175°.

16. The device of claim 14, wherein the first angle is from about 20° to about 70°.

17. The device of claim 14, wherein the cutting edge further comprising a third elongated edge extending from the first elongated edge at a second angle.

18. The device of claim 17, wherein the absolute value of the second angle is substantially equivalent to the absolute value of the first angle.

19. An implant gripping device for detachably holding an implant, wherein the gripping device has a proximal and a distal end, comprising:
a first arm at the distal end;
a second arm at the distal end; wherein the first arm and the second arm are configured to apply a gripping pressure to the implant.
an abutment at the proximal end
a first atraumatic pad between the first arm and the implant

20. The device of claim 19, wherein the first atraumatic pad is between the second arm and the implant.
